(19) United States
(12) Patent Application Publication
(10) Pub. No.: US 2003/0041860 A1
(43) Pub. Date: Mar. 6, 2003

(54) Particulate dispenser

(76) Inventor: Steven N. Verona, Columbus, OH (US)

Correspondence Address: Jason H. Foster, Kremblas, Foster, Phillips & Pollick, 7632 Slate Ridge Blvd., Columbus, OH, 43068, US

(21) Appl. No.: 10/231,754

(22) Filed: Aug. 29, 2002

Related U.S. Application Data
(60) Provisional application No. 60/315,653, filed on Aug. 29, 2001.

(51) Int. Cl. A61M 15/00

(52) U.S. Cl. 128/203.12; 128/203.22

(57) ABSTRACT

A particulate dispenser having a housing with an internal chamber defined by a housing sidewall and endwall. Tobacco snuff, or other particulate such as medication, is contained in the chamber by a closure, which is a circular disk closure rotatably mounted to the housing along its axis. The disk closure has at least one cavity that serves as a measuring cup formed on the substantially planar surface that faces the chamber. By rotating the closure, the cavity filled with particulate is aligned with an air inlet. Thus, when air is inhaled through a nostril near an inhalation port, air is drawn through the air inlet, thereby drawing the particulate through a linear passage and out the inhalation port.

### BACKGROUND OF THE INVENTION

1. Field of the Invention

The invention relates generally to the inhalation of particulate matter, such as medicinal powders and tobacco snuff, and more specifically to a device for containing and aiding in the inhalation of particulate matter such as snuff or medication into a human nostril.

2. Description of the Related Art

When an individual seeks medical treatment it may be that a powdered form of inhalable medicine is prescribed or desired. There are not convenient methods or devices for transporting and inhaling these types of medicines. Therefore, those for whom inhalable medication is necessary have had to make due with prior art devices. Those who wish to inhale other legal powders, such as herbal remedies and tobacco snuff, are likewise left with the prior art.

A prior art device, which is the only conventional means known other than simply sniffing a dose of particulate resting either between two fingers or on a substrate, attempts to solve the problem of aiding in the inhalation of particulate matter. However, because of its design this device has failed to provide the individual with a dosage choice and reliable operation.

The prior art device, known generically as a “snuff dispenser” and shown in FIGS. 17 and 18, operates by first placing the particulate matter in a vial 120 which screws into the base of the main body 100 by internal threads 124 on the main body 100, and external threads on the vial 120. Next, the main body 100 is tilted so that the passageway 104 points downward. The metering lever 130 is rotated so the reservoir 132 faces into the passageway 108 that extends from the vial 120 to the reservoir 132, thereby causing particulate to fall by gravity into the reservoir 132. The lever 130 is then rotated one-quarter turn so that the reservoir faces the interior, cylindrical wall of the main body 100, thereby sealing the reservoir so no particulate falls out in the next step. Next, the main body 100 is tilted upright so that the opening 104 points upward, and then the lever 130 is rotated another quarter turn to position the reservoir 132 containing the particulate facing the opening 104 at the top of the main body 100.

The substance is then inhaled by breathing through the nose while the opening is in close proximity to a nostril. While inhaling, a void 138 allows air to enter the reservoir 132 forcing the particulate into the nose. The particulate matter is forced into the nostril by the air entering from below the particulate matter.

The main problem encountered with this prior art device is that it needs to be cleaned often due to the infiltration of particulate between the lever 130 and the circular cylindrical void in the main body 100 that houses the lever 130. This particulate makes rotation of the lever 130, which is not easy when clean, much more difficult. The device must be cleaned by removing the metering lever, which is difficult to maneuver out of the holding chamber because of the rubber grommet 134, and washing the entire structure. Still further, there is no way to measure more than a single dosage of substance for inhalation. Therefore, there is a need for a particulate dispenser that avoids these problems.

### BRIEF SUMMARY OF THE INVENTION

The invention is a device for containing and aiding in the inhalation of inhalable matter into a human nostril. The invention enables an individual to inhale particulate conveniently and in measured doses, and permits the user to transport a significant amount of particulate in the apparatus, for dispensing at any time desired.

The invention includes a housing having an internal chamber defined by a sidewall and an endwall. The housing has a chamber opening at one housing end and an inhalation port at an opposite, convex housing end. The internal chamber is for storing the particulate matter before dispensing for inhalation, and a closure is movably mounted to the housing over the chamber opening to prevent particulate matter from falling out of the chamber.

An air inlet is formed in the housing adjacent the closure. An elongated, preferably linear fluid passageway extends from the air inlet through the housing to the inhalation port. A cavity is formed in the first surface of the closure, and the cavity has a path of travel that includes the chamber and the air inlet.

There are preferably two cavities on the closure, and these cavities serve as measuring cups, permitting the user to select a precise dose of the particulate matter. By rotating the closure, the cavities are moved from within the chamber, where they can be filled with particulate if the device is oriented appropriately, to the air inlet. When a cavity with particulate matter is positioned at the air inlet, the user can inhale through the nose with the inhalation port at a nostril, thereby causing the particulate to be propelled through the passageway into the nostril.

Because the invention contains two measuring cups with different volumes, the user can choose between a large dose and a small dose. This variation gives the individual more control over the amount of substance inhaled. The design of the device allows for air to flow through the passageway propelling the substance through the passageway and out the inhalation port into the individual's nostril. By rotating the closure clockwise or counterclockwise one can know the exact amount of substance being moved into the inhalation passageway, thus inhalation can take place reliably and with precision.

### BRIEF DESCRIPTION OF THE SEVERAL VIEWS OF THE DRAWINGS

FIG. 1 is a side view illustrating the preferred embodiment of the present invention.

FIG. 2 is a side view in section illustrating the housing portion of the preferred embodiment of the present invention.

FIG. 3 is an end view in section illustrating the housing chamber opening of the embodiment of the present invention.

FIG. 4 is a top view illustrating the closure portion of the preferred embodiment of the present invention.

FIG. 5 is a side view in section through the line 5-5 of FIG. 4, illustrating the closure portion of the preferred embodiment of the present invention.

FIG. 6 is a bottom view illustrating the closure portion of the preferred embodiment of the present invention.

FIG. 7 is a top view illustrating the closure and fastener of the preferred embodiment of the present invention.

FIG. 8 is a top view illustrating the rubber o-ring.

FIG. 9 is a side view illustrating the fastener portion of the preferred embodiment of the present invention.

FIG. 10 is a side view illustrating an alternative embodiment of the present invention.

FIG. 11 is a view in section illustrating the housing portion of the alternative embodiment of the present invention.

FIG. 12 is an end view illustrating the housing chamber opening of the alternative embodiment of the present invention.

FIG. 13 is a side view illustrating the conical lid of the alternative embodiment of the present invention.

FIG. 14 is a top view illustrating the closure and fastener of the alternative embodiment of the present invention.

FIG. 15 is a side view illustrating the fastener portion of the alternative embodiment of the present invention.

FIG. 16 is a side view in section through the line 16-16 of FIG. 14.

FIG. 17 is a side view in section illustrating the metering lever of the prior art.

FIG. 18 is a side view in section illustrating the embodiment of the prior art.

In describing the preferred embodiment of the invention, which is illustrated in the drawings, specific terminology will be used. However, it is not intended that the invention be limited to the specific terms used and it is to be understood that each term includes all technical equivalents, which operate in a similar manner to accomplish a similar purpose. For example, the word connected or term similar thereto is often used. They are not limited to direct connection, but include connection through other elements where such connection is recognized as being equivalent by those skilled in the art.

### DETAILED DESCRIPTION OF THE INVENTION

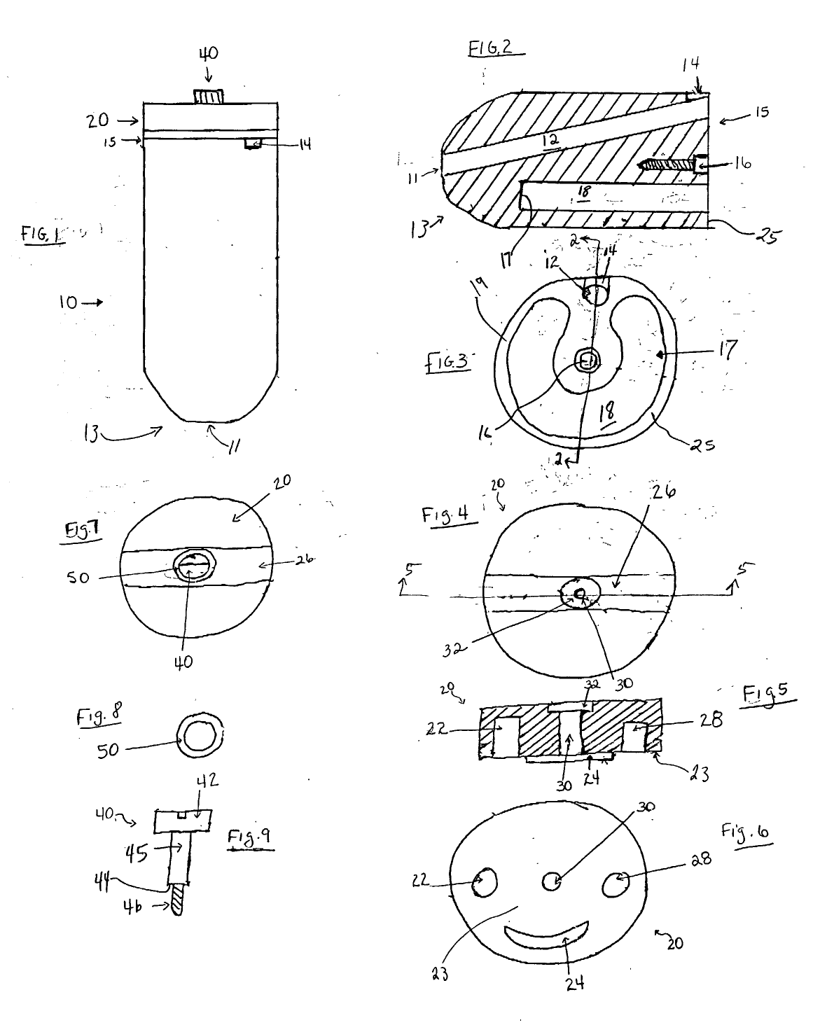

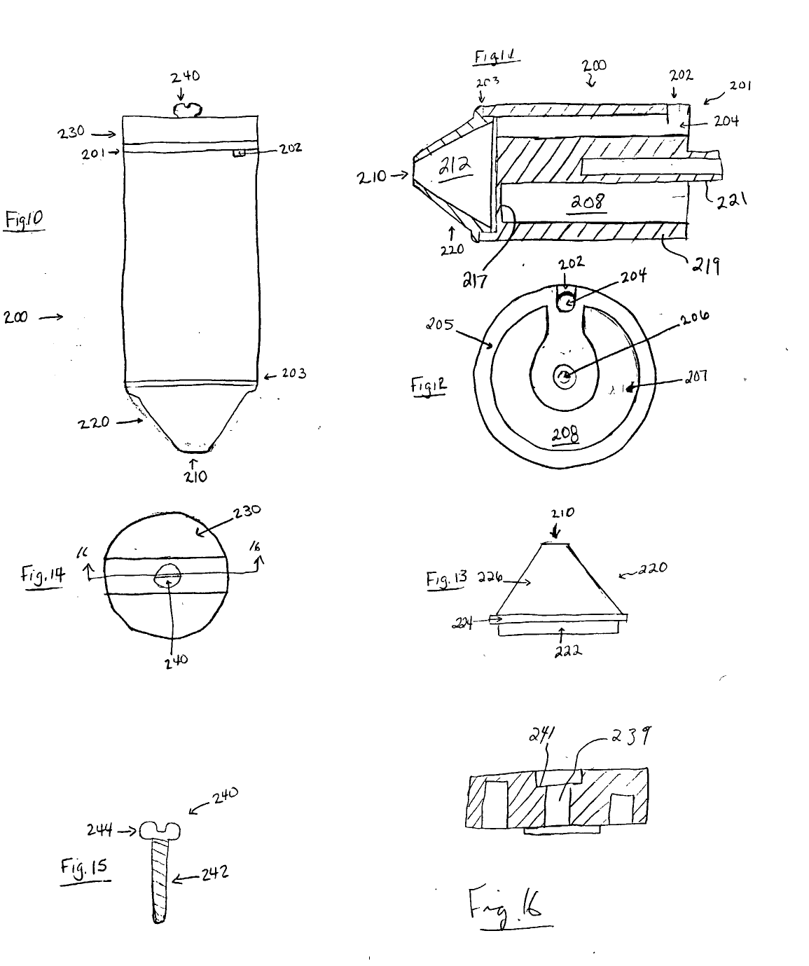

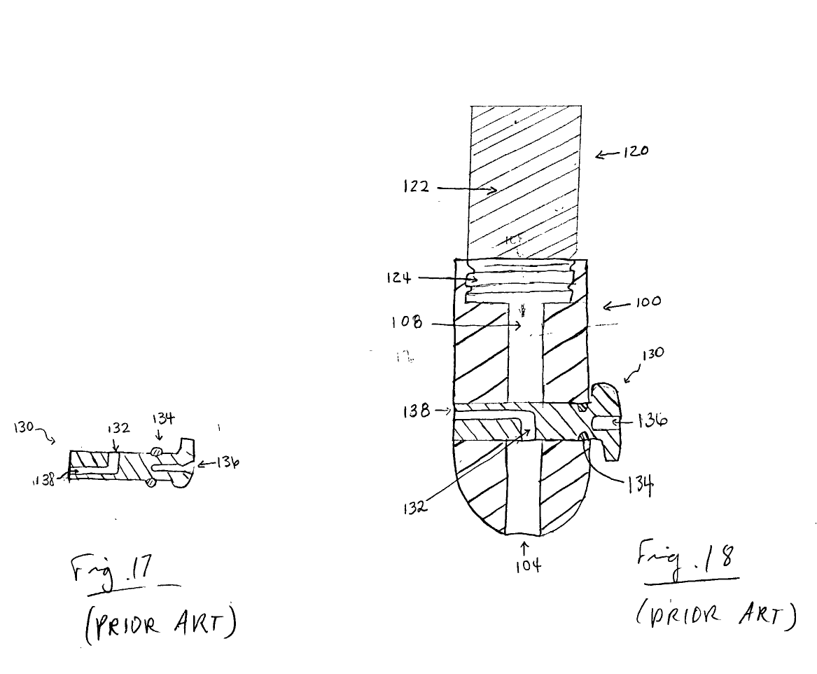

The preferred embodiment of the present invention is shown in FIG. 1. A housing 10 is preferably made of aluminum, however, it may be made from other metals, ceramics, composites or plastics, such as polyvinyl chloride (pvc). The housing 10 has a convex, domed surface at one end 13 for seating against or being inserted within an individual's nostril, and a flat surface at the opposing end 15 for accepting a closure as described further below.

As shown in FIGS. 2 and 3, the housing 10 has a chamber 18 formed therein that is defined by a sidewall 19 and an endwall 17. The chamber 18 can, for example in a preferred embodiment, hold approximately 2 grams of particulate matter. Of course, it will become apparent to the skilled artisan that smaller or larger chambers are also possible for holding smaller or larger amounts of particulate.

The housing 10 is sealed at the opening end 15 by the closure 20. The closure 20 is preferably a plastic disk, but could alternatively be made of any other material, such as metal, ceramic or a composite. The closure 20 has a substantially planar surface 23 that seats against the substantially planar surface 25 at the opening end 15 of the housing 10.

The closure 20 is movably mounted to the housing 10, meaning the closure can be moved relative to the housing 10 by manipulation by an average person. Preferably, the closure is rotatably mounted by a fastener 40, such as a screw, to the housing, thereby permitting rotation of the closure around the fastener 40. The preferred structure for rotatably mounting the closure 20 to the housing 10 is described next, and it is understood that alternative structures, which cannot possibly all be described herein, could accomplish the same function.

The fastener 40 is preferably a shoulder screw that threads into the receptacle 16, which is shown in FIG. 2 to be a female, threaded void formed in the housing 10. As shown in FIG. 9, the screw has a head 42, a cylindrical shank 45 that terminates in a shoulder 44, and a threaded shaft 46. Referring to FIG. 8, a bias, preferably the rubber o-ring 50, is positioned between the head 42 of the fastener 40 and the lip 32 (see FIG. 5) when the shank 45 extends through the passage 30 of the closure 20. The shoulder 44 of the fastener 40 seats against the corresponding seat in the receptacle 16 (shown in FIG. 2) before the head 42 of the fastener 40 seats too firmly against the o-ring 50. Thus, by tightening the o-ring 50 gently between the head 42 and the lip 32 of the closure at the same time that the shoulder 44 seats firmly against the corresponding seat in the receptacle 16, the closure is held firmly in place by the bias of the o-ring 50, but not so firmly that rotation is restricted significantly. Furthermore, the wall of the passage 30 of the closure simply rotates relative to the shank 45 of the fastener, thereby causing the shank 45 to serve as an axle. Therefore, the fastener 40 also does not tend to become tightened or loosened by rotation of the closure 20.

The closure has a grippable contour 26 to permit a user to conveniently rotate the closure 20 by manual manipulation, for example, between the thumb and forefinger. Furthermore, by manually unscrewing the fastener 40, the closure 20 can be removed from the housing 10, thereby exposing the chamber opening for the addition or removal of particulate matter.

Referring to FIGS. 2 and 3, an elongated, preferably linear passageway 12 extends through the housing 10. The passageway 12 extends from the inhalation port 11, which is formed at the domed end 13, to the air inlet 14, which is formed near, and preferably at, the opening end 15 of the housing 10. The passageway 12 is angled relative to the housing's axis to permit the preferably linear passageway 12 to connect the inhalation port 11, which preferably lies on the housing's axis, and the air inlet 14, which preferably lies at the radially outermost part of the housing 10. The angle allows for unobstructed, linear movement of the particulate matter through the passageway 12 and into the individual's nostril.

Referring to FIGS. 5 and 6, the closure 20 contains two measuring cavities, preferably the cups 22 and 28. Of course, only one cavity, or more than two cavities, could be formed in the closure 20. The cups 22 and 28 are formed in the substantially planar surface 23 that faces the chamber 18 when the closure is in its operable position rotatably mounted to the housing 10. In this operable position, at least one, and frequently both, cups 22 and 28 are positioned within the chamber 18, thereby permitting filling of the cups 22 and 28 with particulate under the influence of gravity. The cups 22 and 28 are positioned in the chamber 18, because the chamber 18 is formed around the majority of the housing 10, as shown in FIG. 3, and the cups 22 and 28 are at opposite sides of the circular closure 20. Thus, at least one, and possibly both, of the cups 22 and 28 (at 180 degrees from one another) are within the chamber 18, which extends nearly 360 degrees around the circular cylindrical housing 10.

When the inhalation port 11 points upwardly, either or both of the cups 22 and 28 within the chamber 18 receive by gravitational force some of the particulate matter contained therein, thereby permitting the particulate to be dispensed in predetermined amounts based upon the volume of the cups 22 and 28. The cups 22 and 28 are not of equal volume, for example one could be twice as large as the other, thus giving the individual the option to choose a larger or smaller dose.

An arcuate alignment guide 24 extends from the substantially planar surface 23, and provides seats to limit the rotation of the closure 20. The alignment guide 24 is always disposed in the chamber 18 when the closure 20 is in the operable position, and the opposite ends thereof seat against the sidewall 19 of the chamber 18 at the extreme positions of closure 20 rotation. Thus, rotation of the closure 20 to one extreme causes one end of the alignment guide 24 to seat against the sidewall 19, thereby aligning one cup with the air inlet 14. Rotation of the closure 20 to the other extreme causes the opposite end of the alignment guide 24 to seat against the sidewall 19, thereby aligning the other cup with the air inlet 14. And when a cup filled with particulate is aligned with the air inlet 14, and the opening 11 is placed near the nostril, inhalation causes a negative pressure in the passageway 12, which draws air into the air inlet, thereby sweeping the particulate in the cup into the passageway and into the nostril.

When the closure 20 is turned clockwise, the larger cup is moved toward the air inlet 14, and a large dose can be inhaled. When turned counterclockwise, a small dose is dispensed. Thus, the user has a quick reference as to how much of the particulate matter will be inhaled. And the particulate in each cup is leveled off when the cup passes across the surface 25, thereby dispensing a precisely measured quantity of particulate in the cup.

As described above, when inhalation occurs a negative pressure is created at the inhalation port 11. This causes air to enter the air inlet 14, which causes eddie currents to remove the particulate matter from the cup aligned in the air inlet 14, force it up through the fluid passageway 12 and out the inhalation port 11 into the nostril. In the embodiment described above, the air does not force the particulate matter up through the fluid passageway 12, as would be the case if the air were entering from below the particulate matter. Instead, air is moving across the top of the cup and its particulate matter, thereby causing the matter to swirl and be pulled through the fluid passageway 12 and into the individual's nostril. However, small holes can be formed in the closure 20 at the cups to promote the flow of air through the cup, and not just over it.

An alternative embodiment of the present invention is shown in FIG. 10. This embodiment functions under the same principles as the device shown in FIG. 1, however, there are some structural differences.

The housing 200 has a substantially circular cylindrical exterior surface, and an internal chamber 208 defined by the sidewall 219 and the endwall 217. Much the same as the embodiment shown in FIG. 1, the chamber 208 houses particulate matter, and the housing 200 has an opening end 201 and an opposite, lid end 203.

A convex lid 220, which is substantially conical in external shape, mounts to the housing 200 at the lid end 203. The lid 220 is removably mounted to the housing 200 by a lip that frictionally engages, and inserts within, a rim of the housing 200, as shown in FIG. 11. A substantially conically shaped chamber 212 is formed within the lid 220.

A fluid passageway 204 begins at the substantially planar opening end 201 of the housing 200, and follows a substantially linear path through the housing 200 and opens into the conical chamber 212, which is in fluid communication with an inhalation port 210. The passageway 204 is preferably substantially parallel to the axis of the substantially cylindrical housing 200.

The closure 230, which has a similar configuration to the closure 20, mounts to the housing 200 with the housing shaft 221 extending through the passage 239. The head 244 of the screw 240 seats against the end of the shaft 221 and the shoulder 241 when the threaded end of the screw is inserted within the passage in the shaft 221. The diameter of the head 244 is larger than the diameter of the passage 239, and therefore the closure 230 is held in place slightly differently than the closure 20.

Air drawn in through the air inlet 202 pulls the particulate matter through the fluid passageway 204, into the chamber 212 and out the inhalation port 210. The housing 200 has an internal storage chamber 208 that can hold approximately 2.5 grams of particulate matter, and it will become apparent that this amount could be varied.

While certain preferred embodiments of the present invention have been disclosed in detail, it is to be understood that various modifications may be adopted without departing from the spirit of the invention or scope of the following claims.

1. A device for containing and aiding in the inhalation of inhalable matter into a human nostril, the device comprising:
a) a housing having an internal chamber defined by a sidewall and an endwall, said housing having a chamber opening at one housing end and an inhalation port at an opposite, convex housing end;
b) a closure movably mounted to the housing over the opening, the closure having a first, substantially planar surface seating against the housing sidewall at the chamber opening;
c) an air inlet formed in the housing adjacent the closure;
d) an elongated fluid passageway extending through the housing from the air inlet to the inhalation port; and
e) at least one cavity formed in the first surface of the closure, said cavity having a path of travel that includes the chamber and the air inlet.

2. The device in accordance with claim 1, wherein the closure is rotatably mounted to the housing.

3. The device in accordance with claim 1, further comprising a reservoir on the opposite side of the chamber's endwall, defined by a conical lid that forms the convex housing end.

4. The device in accordance with claim 1, wherein the fastener has a head on one end, a substantially cylindrical shoulder and a threaded end which seats into the housing.

5. The device in accordance with claim 1, further comprising a grippable contour on the closure.

6. The device in accordance with claim 1, further comprising an arcuate alignment guide extending from the substantially planar surface of the closure.

7. The device in accordance with claim 1, further comprising a plurality of cavities on the closure.

8. The device in accordance with claim 1, wherein the fastener is a screw.

9. The device in accordance with claim 1, wherein a bias seats around the shoulder and against the head of the fastener.

10. The device in accordance with claim 9, wherein the bias is an o-ring.

11. A device for containing and aiding in the inhalation of inhalable matter into a human nostril, the device comprising:
a) a housing having an internal chamber defined by a sidewall and an endwall, said housing having a chamber opening at one housing end and an inhalation port at an opposite, convex housing end;
b) a closure rotatably mounted to the housing over the opening, the closure having a first, substantially planar surface seating against the housing sidewall at the chamber opening;
c) an air inlet formed in the housing adjacent the closure; an elongated fluid passageway extending through the housing from the air inlet to the inhalation port; and
d) at least one cavity formed in the first surface of the closure, said cavity having a path of travel that includes the chamber and the air inlet.

12. The device in accordance with claim 10, further comprising a reservoir on the opposite side of the chamber's endwall, defined by a conical lid that forms the convex housing end.

13. The device in accordance with claim 12, wherein the fastener has a head on one end, a substantially cylindrical shoulder and a threaded end which seats into the housing.

14. The device in accordance with claim 13, further comprising a grippable contour on the closure.

15. The device in accordance with claim 14, further comprising an arcuate alignment guide extending from the substantially planar surface of the closure.

16. The device in accordance with claim 15, further comprising a plurality of cavities on the closure.

17. The device in accordance with claim 16, wherein the fastener is a screw.

18. The device in accordance with claim 17, wherein a bias seats around the shoulder and against the head of the fastener.
